(19) United States
(12) Patent Application Publication
(10) Pub. No.: US 2021/0348106 A1
(43) Pub. Date: Nov. 11, 2021

(54) GAS DISSOLUTION DEVICE AND ALGAE CULTIVATION DEVICE

(71) Applicant: USUI CO., LTD., Shizuoka (JP)

(72) Inventors: Toshifumi KASHIWAGI, Sunto-gun, Shizuoka (JP); Iwane SUZUKI, Tsukuba-shi, Ibaraki (JP)

(21) Appl. No.: 17/284,926

(22) PCT Filed: Oct. 23, 2019
(86) PCT No.: PCT/JP2019/041493

§ 371 Date: Apr. 13, 2021

(30) Foreign Application Priority Data
Oct. 26, 2018 (JP) 2018-201899

(51) Int. Cl. C12M 1/00; C12M 1/34; B01F 3/04; B01F 5/10; B01F 15/00

(52) U.S. Cl. CPC C12M 29/06; C12M 41/44; B01F 3/04113; B01F 2215/0073; B01F 15/00422; B01F 15/0022; B01F 2003/04893; B01F 5/10

(57) ABSTRACT

The gas dissolution device includes a dissolution vessel storing a part of culture solution in a culture vessel, a gas supply pipe connected to a carbon dioxide cylinder and supplying carbon dioxide through an end inserted into the dissolution vessel, a gas discharger provided at the gas supply pipe and turning the carbon dioxide into microbubbles, and a mass flow controller controlling a flowrate of the carbon dioxide flowing in the gas supply pipe. Here, a water depth from the gas discharger to a liquid level of the culture solution in the dissolution vessel is set deeper than a water depth of the culture solution in the culture vessel.

### TECHNICAL FIELD

This disclosure relates to a gas dissolution device and an algae cultivation device.

### BACKGROUND

A gas dissolution device has been known (e.g., Patent Literature 1). The conventional gas dissolution device supplies a part of culture solution stored in a culture vessel to a dissolution vessel together with high-concentration carbonic acid gas, and quickly dissolves the carbonic acid gas in the culture solution by generating a turbulent flow in the dissolution vessel. The culture solution in which the carbonic acid gas has been dissolved is then returned to the culture vessel from the dissolution vessel.

### CITATION LIST

### Patent Literature

Patent Literature 1: JP 1994-153912 A

### SUMMARY

### Technical Problem

The conventional gas dissolution device dissolves carbonic acid gas in the dissolution vessel rapidly and with high concentration in response to the shortage of the carbonic acid gas in the culture vessel, and the culture solution in which the carbonic acid gas has been dissolved is returned to the culture vessel. With this, the concentration of carbonic acid gas in the culture solution recovers rapidly. However, there is a concern that the high-concentration carbonic acid gas which has been dissolved in the culture solution may be released into the atmosphere easily due to the turbulent flow generated by continuous stirring of the culture solution. Additionally, although the carbonic acid gas is supplied from a carbonic acid gas source, it is desirable from the environmental and economic aspects that the carbonic acid gas has a large dissolved amount with respect to the input amount. That is, an index of dissolution efficiency, i.e., a ratio of dissolved amount to input amount, can be used for evaluation, and it is desirable to achieve a high dissolution efficiency.

The present disclosure has been made in view of the above circumstances, and an object of the present disclosure is to provide a gas dissolution device and an algae cultivation device capable of improving a gas dissolution efficiency with respect to liquid.

### Solution to Problem

In order to achieve the above object, a gas dissolution device of the present disclosure comprises a dissolution vessel that stores a part of liquid stored in a main vessel, a gas supply pipe that is connected to a gas supply source and supplies gas through a tip part inserted into the liquid stored in the dissolution vessel, a gas discharger that is provided at the tip part of the gas supply pipe and turns the gas supplied from the tip part into bubbles, and a gas controller that controls a flowrate of the gas flowing in the gas supply pipe. A water depth from the gas discharger to a liquid level of the liquid stored in the dissolution vessel is set deeper than a water depth of the liquid stored in the main vessel.

Additionally, in order to achieve the above object, an algae cultivation device of the present disclosure comprises a culture vessel that stores culture solution for culturing algae, and a gas dissolution device that dissolves carbon dioxide in the culture solution. The gas dissolution device comprises a dissolution vessel that stores a part of the culture solution stored in the culture vessel, a first circulation pipe and a second circulation pipe that communicate between the culture vessel and the dissolution vessel, a first pump that delivers the culture solution stored in the culture vessel to the dissolution vessel through the first circulation pipe, a second pump that returns the culture solution stored in the dissolution vessel to the culture vessel through the second circulation pipe, a gas supply pipe that is connected to a carbon dioxide source and supplies carbon dioxide through a tip part inserted into the culture solution stored in the dissolution vessel, a gas discharger that is provided at the tip part of the gas supply pipe and turns the carbon dioxide supplied from the tip part into bubbles, and a gas controller that controls a flowrate of the carbon dioxide flowing in the gas supply pipe. A water depth from the gas discharger to a liquid level of the culture solution stored in the dissolution vessel is set deeper than a water depth of the culture solution stored in the culture vessel.

### Advantageous Effects

With the gas dissolution device and the algae cultivation device of the present disclosure, it is possible to improve the gas dissolution efficiency with respect to liquid.

### BRIEF DESCRIPTION OF DRAWINGS

FIG. 1 is an overview configuration diagram showing an algae cultivation device of a first embodiment.

FIG. 2 is a configuration diagram showing a gas dissolution device of the first embodiment.

FIG. 3A is a table showing relations of pore diameters of a gas discharger, class values (bubble sizes) of sphere-equivalent diameters of bubbles at gas flowrate of 10 mL/min, and average numbers of the bubbles generated at gas flowrate of 10 mL/min in the gas dissolution device of the first embodiment.

FIG. 3B is a graph showing the relations between the pore diameters of the gas discharger and the class values (bubble sizes) of sphere-equivalent diameters of the bubbles at gas flowrate of 10 mL/min in the gas dissolution device of the first embodiment.

FIG. 3C is a graph showing the relations between the pore diameters of the gas discharger and the average numbers of the bubbles generated at gas flowrate of 10 mL/min in the gas dissolution device of the first embodiment.

FIG. 4A is a graph showing the relations between the class values (bubble sizes) of sphere-equivalent diameters of the bubbles and dissolution efficiency of carbon dioxide in the gas dissolution device of the first embodiment.

FIG. 4B is a graph showing the relations between water depths from the gas discharger to liquid levels in a dissolution vessel and the dissolution efficiencies of carbon dioxide in the gas dissolution device of the first embodiment.

FIG. 5 is a graph showing the relations between the water depths from the gas discharger to the liquid levels in the dissolution vessel and the dissolution efficiencies of carbon dioxide in the gas dissolution device of the first embodiment when the bubble size is 1.4 mm.

FIG. 6 is a table showing bubble sizes of bubbles generated at gas flowrate of 10 mL/min in the gas dissolution device of the first embodiment when the pore diameters of the gas discharger is 2 μm.

FIG. 7 is a table showing the relations between the pore diameters of the gas discharger and a number of bubbles passing through a cross-sectional area per unit time in the gas dissolution device of the first embodiment.

FIG. 8 is a table showing relations of the pore diameters of the gas discharger, the water depths from the gas discharger to the liquid levels in the dissolution vessel, liquid amounts in the dissolution vessel, actual dissolved amounts of carbon dioxide, and the dissolution efficiencies of carbon dioxide in the gas dissolution device of the first embodiment.

FIG. 9A is a table showing results of carbon dioxide dissolution experiments with the gas dissolution device of the first embodiment, a first comparative example, and a second comparative example.

FIG. 9B is a graph showing the results of carbon dioxide dissolution experiments with the gas dissolution device of the first embodiment, the first comparative example, and the second comparative example.

### DESCRIPTION OF EMBODIMENTS

Hereinafter, an embodiment for implementing a gas dissolution device and an algae cultivation device of the present disclosure will be described with reference to the accompanying drawings.

### First Embodiment

An algae cultivation device 1 of the first embodiment is a device for artificially culturing microalgae and includes a culture vessel 2 and a gas dissolution device 3.

It should be understood that algae herein mean photosynthetic organisms that generate oxygen excluding moss plants, fern plants, and seed plants, and collectively refers to plants with photosynthetic pigments that live in water. Algae are excellent in growth, have a high yield per area, and accumulate a large amount of useful substances such as fats and oils. Hence, algae have a high utility value so as to be used as raw materials for health foods, supplements, chemical raw materials, biofuels, etc. The microalgae cultivated by the algae cultivation device 1 are unicellular algae having a body length of several μm to several hundred and are of a size that the individual existence is hardly visible by a human naked eye. The examples of the microalgae include green algae such as Spirulina, Euglena, Chlorella, Dunaliella salina, and Botryococcus.

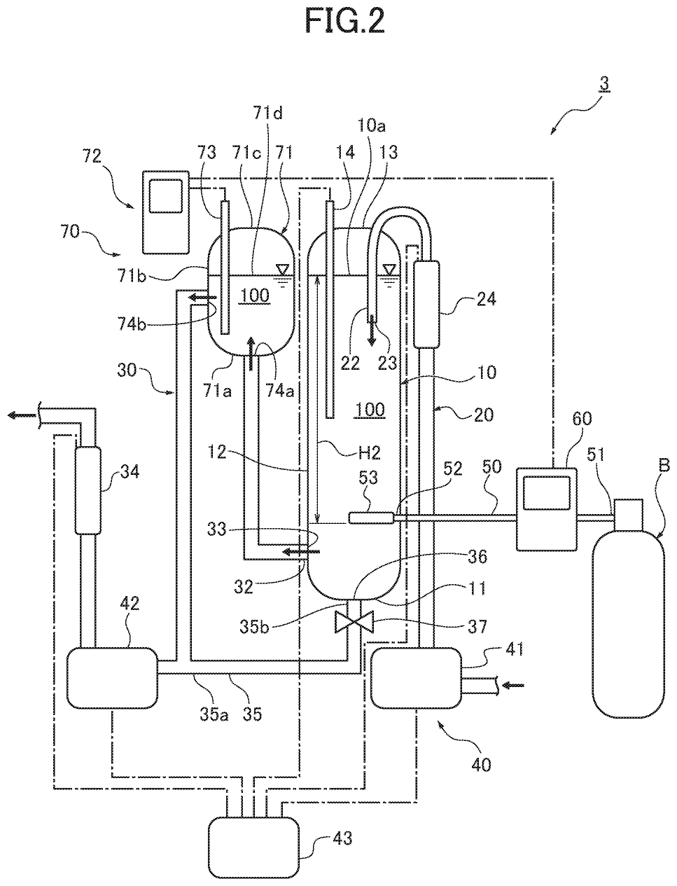

The culture vessel 2 (main vessel) is a water vessel that stores culture solution 100 (liquid) in which microalgae are suspended. The culture vessel 2 shown in FIG. 2 is a raceway type vessel with an oblong circulation channel. The culture vessel 2 has an opening 2a opened upward such that the culture solution 100 stored therein is exposed to the outside air. The culture solution 100 stored in the culture vessel 2 has a liquid volume of 150 L and a water depth H1 of 130 to 135 mm. The culture vessel 2 is equipped with a waterwheel 4, and the culture solution 100 is stirred at a stirring speed of 11 cm/sec by the rotation of the waterwheel 4. Here, the “water depth H1” represents a distance from the bottom surface of the culture vessel 2 to the liquid level of the culture solution 100 stored in the culture vessel 2. It should be noted that the volume and/or the shape of the culture vessel 2 are not limited to the above configuration and are selectively determined in accordance with a type of the algae to be cultivated and/or a cultivation method to be used.

The gas dissolution device 3 controls a carbon dioxide concentration of the culture solution 100 in the culture vessel 2. To that end, the gas dissolution device 3 dissolves carbon dioxide (gas) in a part of the culture solution 100 retrieved from the culture vessel 2, and returns the culture solution 100 in which the carbon dioxide has been dissolved to the culture vessel 2. Here, inputting an excess amount of carbon dioxide into the culture solution 100 may inhibit the culture of microalgae. On the other hand, it is possible to rapidly culture the microalgae which is rich in useful substances by appropriately controlling the carbon dioxide concentration of the culture solution 100.

The gas dissolution device 3 is installed on a trolly with wheels (not illustrated) and includes a dissolution vessel 10, a first circulation pipe 20, a second circulation pipe 30, a circulation mechanism 40, a gas supply pipe 50, a mass flow controller 60 (gas controller), and a pH monitor 70.

The dissolution vessel 10 is a vessel for storing a part of the culture solution 100 retrieved from the culture vessel 2 and dissolving carbon dioxide into the stored culture solution 100. The dissolution vessel 10 has a vertically extended tube shape having a bottom surface 11 and side surface 12, and the upper part of the dissolution vessel 10 is closed by an upper surface 13. In this embodiment, the bottom surface 11 is a curved surface, and the upper surface 13 is covered by a non-sealed lid.

The side surface 12 of the dissolution vessel 10 has a sufficient height such that a water depth H2 from a gas discharger 53 described later to a liquid level 10a of the culture solution 100 stored in the dissolution vessel 10 is adjustable to be deeper than the water depth H1 in the culture vessel 2. The liquid amount of the culture solution 100 stored in the dissolution vessel 10 is set to equal to or less than one-twentieth of the liquid amount of the culture solution 100 stored in the culture vessel 2, and in this embodiment, is set to 5 liters. A liquid level sensor 14 is installed on the upper surface 13 of the dissolution vessel 10 to monitor the amount of the culture solution 100 stored therein. The detected values of the liquid level sensor 14 are input to a pump controller 43 of the circulation mechanism 40.

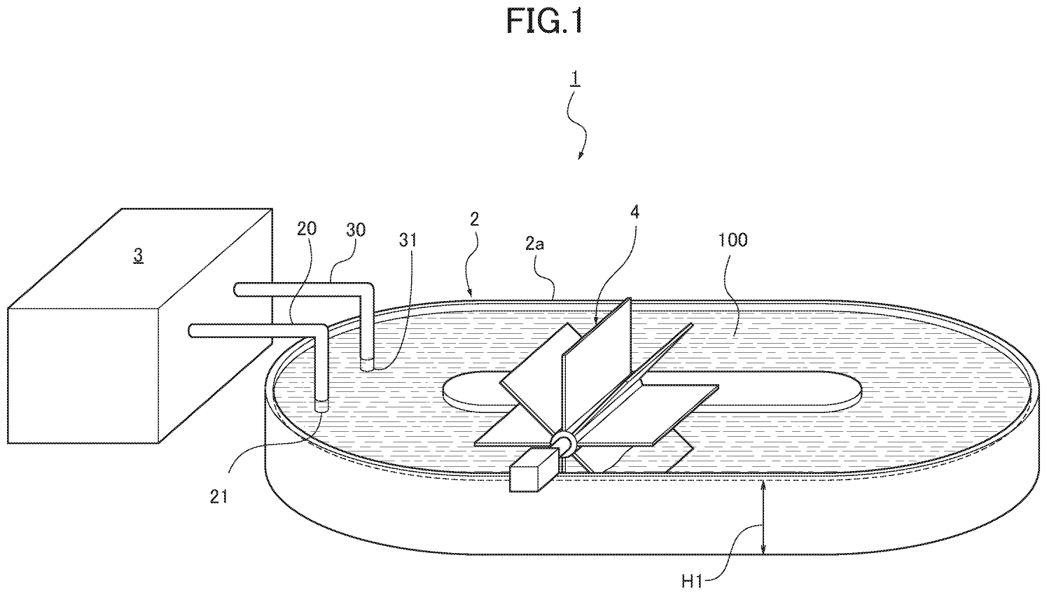

The first circulation pipe 20 communicates the culture vessel 2 and the dissolution vessel 10 such that the culture solution 100 to be delivered from the culture vessel 2 to the dissolution vessel 10 passes through the first circulation pipe 20. As shown in FIG. 1, a one end 21 of the first circulation pipe 20 is inserted into the culture solution 100 stored in the culture vessel 2. The other end 22 of the first circulation pipe 20 penetrates the upper surface 13 of the dissolution vessel 10 and is inserted inside the dissolution vessel 10. A liquid ejection port 23 is formed at the other end 22 of the first circulation pipe 20. The liquid ejection port 23 is provided at a position higher than the gas discharger 53 and is oriented toward the bottom surface 11 of the culture vessel 2.

In the first circulation pipe 20, a first flowmeter 24 and a first pump 41 of the circulation mechanism 40 are provided. The first flowmeter 24 is placed downside of the first pump 41. With the first flowmeter 24, the flowrate of the culture solution 100 which is discharged by the first pump 41 and delivered into the dissolution vessel 10 is detected. The detected values of the first flowmeter 24 are input to the pump controller 43 of the circulation mechanism 40.

The second circulation pipe 30 communicates the culture vessel 2 and the dissolution vessel 10 such that the culture solution 100 to be returned from the dissolution vessel 10 to the culture vessel 2 passes through the second circulation pipe 30. As shown in FIG. 1, a one end 31 of the second circulation pipe 30 is inserted into the culture solution 100 stored in the culture vessel 2. The other end 32 of the second circulation pipe 30 is connected to the side surface 12 of the dissolution vessel 10. A liquid suction port 33 is formed at the other end 32 of the second circulation pipe 30 and is open to the side surface 12. The liquid suction port 33 is provided at a position lower than the gas discharger 53.

In the middle of the second circulation pipe 30, a monitoring vessel 71 of the pH monitor 70, a second flowmeter 34, and a second pump 42 of the circulation mechanism 40 are provided. The monitoring 71 is placed in the uppermost stream, and the second pump 42 and the second flowmeter 34 are placed downstream of the monitoring vessel 71 in this order. With the second flowmeter 34, the flowrate of the culture solution 100 which is discharged by the second pump 42 and returned to the culture vessel 2 is detected. The detected values of the second flowmeter 34 are input to the pump controller 43 of the circulation mechanism 40.

Further, a one end 35a of a discharge pipe 35 is connected to a part of the second circulation pipe 30 between the monitoring vessel 71 and the second pump 42. The discharge pipe 35 is a pipe to return the culture solution 100 discharged from the dissolution vessel 10 to the culture vessel 2 while bypassing the monitoring vessel 71. The other end 35b of the discharge pipe 35 is connected to a liquid discharge opening 36 formed on the bottom surface 11 (bottom part) of the dissolution vessel 10. Accordingly, the culture solution 100 stored in the dissolution vessel 10 flows into the discharge pipe 35 through the liquid discharge opening 36. Further, a switching valve 37 is provided in the discharge pipe 35. The switching valve 37 is a normally closed valve and allows the culture solution 100 in the discharge pipe 35 to directly flow into the second circulation pipe 30 when open. The opening and closing of the switching valve 37 are operated manually.

The circulation mechanism 40 delivers a part of the culture solution 100 stored in the culture vessel 2 to the dissolution vessel 10 through the first circulation pipe 20 and returns the culture solution 100 stored in the dissolution vessel 10 to the culture vessel 2 through the second circulation pipe 30. In this embodiment, the circulation mechanism 40 continuously circulates the culture solution 100 between the culture vessel 2 and the dissolution vessel 10. The circulation mechanism 40 includes the first pump 41, the second pump 42, and the pump controller 43.

The first pump 41 is provided in the first circulation pipe 20. The first pump 41 is a magnet pump which sucks and discharges the culture solution 100 in the culture vessel 2 to deliver the culture solution 100 from the culture vessel 2 to the dissolution vessel 10. The second pump 42 is provided in the second circulation pipe 30. The second pump 42 is a magnet pump which sucks and discharges the culture solution in the dissolution vessel 10 to deliver the culture solution 100 from the dissolution vessel 10 to the culture vessel 2. In this embodiment, the first pump 41 has better performance than the second pump 42.

It should be noted that the first pump 41 may be set to have similar performance to the second pump 42. In such a case, a mechanism for adjusting the output may be provided in order to adjust the performance of the pumps similar to each other. It should also be noted that the first pump 41 and the second pump 42 are not limited to magnet pumps but may be diaphragm pumps or turbo pumps such as centrifugal pumps, mixed flow pumps and axial flow pumps.

The pump controller 43 controls the operation of the first pump 41 and the second pump 42 to circulate one-twentieth or less of the liquid amount (in this embodiment, 1 to 2 liters) of the culture solution 100 stored in the culture vessel 2. The pump controller 43 includes a Central Processing Unit (CPU), a memory, and the like, and the detected values of the liquid level sensor 14, the detected values of the first flowmeter 24, and the detected values of the second flowmeter 34 are input to the pump controller 43. The pump controller 43 controls the operation of the first and second pumps 41, 42 to maintain a constant liquid amount of the culture solution 100 stored in the dissolution vessel 10 based on the detected values of the liquid level sensor 14. Additionally, the pump controller 43 controls the operation of the first pump 41 and the second pump 42 based on the detected values of the first flowmeter 24 and the second flowmeter 34, such that the flowrate of the culture solution 100 which is discharged by the first pump 41 in the first circulation pipe 20 and the flowrate of the culture solution 100 which is discharged by the second pump 42 in the second circulation pipe 30 become equal to each other.

The gas supply pipe 50 is a pipe through which the carbon dioxide (gas) is input to the dissolution vessel 10 from a carbon dioxide cylinder B (gas source or carbon dioxide source). A one end 51 of the gas supply pipe 50 is connected to the carbon dioxide cylinder B. The other end 52 (tip part) of the gas supply pipe 50 penetrates the side surface 12 of the dissolution vessel 10 and is inserted inside the culture solution 100 stored in the dissolution vessel 10. Further, the gas discharger 53 is fixed to the other end 52 of the gas supply pipe 50 in the dissolution vessel 10.

The gas discharger 53 turns the carbon dioxide supplied from the gas supply pipe 50 into fine bubbles (e.g., microbubbles and nanobubbles. Hereinafter, collectively referred to as “bubbles”) inside the culture solution 100 stored in the dissolution vessel 10. In this embodiment, the gas discharger 53 has a cylindrical shape and is formed of a porous ceramic material, a sintered alloy, a polymer compound, or the like. The pore diameters of the gas discharger 53 is 1 to 100 μm.

The gas discharger 53 generates bubbles having a sphere-equivalent diameter of 2.5 mm or smaller, preferably 1.0 mm or smaller. The number of bubbles which are generated by the gas discharger 53 and pass through a unit cross-section area per unit time is 35/min/cm² or more.

The gas discharger 53 is arranged at a position such that the water depth H2 to the liquid level 10a of the culture solution 100 stored in the dissolution vessel 10 (i.e., depth from gas discharger 53 to liquid level 10a) is deeper than the water depth H1 of the culture solution 100 stored in the culture vessel 2. In this embodiment, the gas discharger 53 is positioned such that the water depth H2 becomes 450 mm or deeper. However, in case where the gas discharger 53 generates bubbles having the sphere-equivalent diameter of 1.4 mm or smaller, the water depth H2 may be set to 350 mm or deeper.

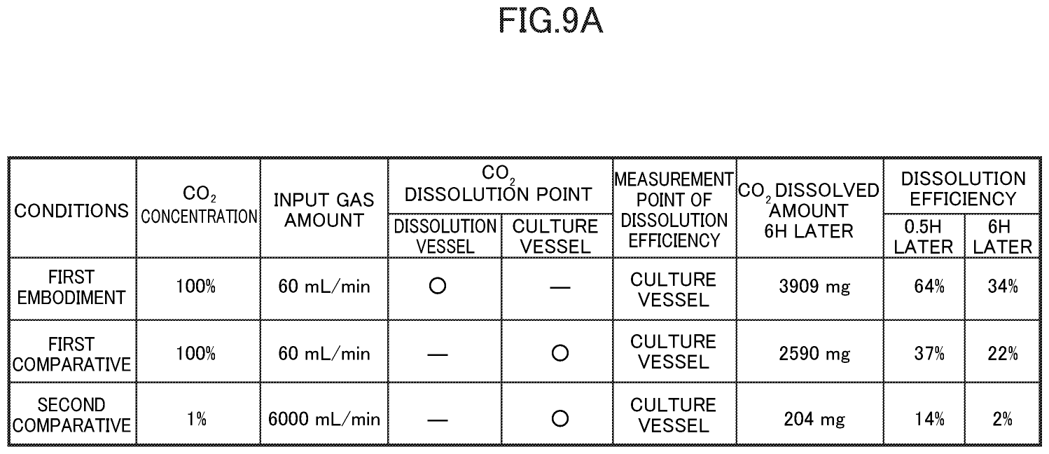

In the gas dissolution device 3, the pore diameter of the gas discharger 53, the water depth H2, as well as the liquid amount and the setting values of the dissolution vessel 10 are adjusted in order to control the dissolved amount of carbon dioxide in the culture solution 100 stored in the dissolution vessel 10 to be 200 mg/L or less in terms of dissolved inorganic carbon weight. FIG. 8 shows the examples of the pore diameters of the gas discharger 53, the water depths H2, as well as the liquid amounts and setting values of the dissolution vessel 10 that achieve the dissolved amount of carbon dioxide in the culture solution 100 in the dissolution vessel 10 to be 200 mg/L or less in terms of dissolved inorganic carbon weight.

The mass flow controller 60 measures the flowrate of carbon dioxide flowing in the gas supply pipe 50 and controls the flowrate of carbon dioxide. The mass flow controller 60 receives a control command from a pH controller 72 provided in the pH monitor 70. The mass flow controller 60 controls the flowrate of carbon dioxide flowing in the gas supply pipe 50 based on the control command from the pH controller 72.

The pH monitor 70 monitors a pH value of the culture solution 100 stored in the dissolution vessel 10. The pH monitor 70 includes the monitoring vessel 71, the pH controller 72, and a pH sensor 73.

The monitoring vessel 71 is provided in the middle of the second circulation pipe 30. The monitoring vessel 71 communicates with the dissolution vessel 10 through the second circulation pipe 30 and stores a part of the culture solution 100 flowing out of the dissolution vessel 10. The monitoring vessel 71 has a vertically extended tube shape having a bottom surface 71a and a side surface 71b, and the upper part of the monitoring vessel 71 is closed by an upper surface 71c. In this embodiment, the bottom surface 71a is a curved surface, and the upper surface 71c is covered by a non-sealed lid. The liquid amount of the culture solution 100 stored in the monitoring vessel 71 can be arbitrary determined, and in this embodiment, set to 1 liter.

An inlet 74a through which the culture solution 100 is flown into the monitoring vessel 71 via the second circulation pipe 30 is formed on the bottom surface 71a. An outlet 74b through which the culture solution 100 is flown out of the monitoring vessel 71 is formed on the side surface 71b. The monitoring vessel 71 is positioned such that the height of the liquid level 71d of the culture solution 100 stored in the monitoring vessel 71 is equal to the height of the liquid level 10a of the culture solution 100 stored in the dissolution vessel 10.

The pH controller 72 includes, for example, a Central Processing Unit (CPU) and a memory, and the detected values of the pH sensor 73 are input to the pH controller 72. The pH controller 72 outputs the control command to the mass flow controller 60 based on the detected value of the pH sensor 73 such that the pH value falls within an appropriate range according to a required dissolved amount of carbon dioxide for algae cultivation. For example, the pH controller 72 outputs a control command to stop the inflow of carbon dioxide to the dissolution vessel 10 when the detected pH value becomes a predetermined value or less.

The pH sensor 73 is installed on the upper surface 71c of the monitoring vessel 71, and the sensor part thereof is inserted into the culture solution 100 stored in the monitoring vessel 71. The pH value of the culture solution 100 stored in the monitoring vessel 71 is detected by the pH sensor 73.

A problem of a conventional gas dissolution device will be explained below.

In algae cultivation, it is important to control the carbon dioxide concentration of culture solution within an appropriate range, as described above. In conventional devices, the dissolution of carbon dioxide in culture solution is generally performed by directly inserting an air diffusing tube (air diffuser) into the culture solution. However, when the air diffusing tube is directly inserted into the culture solution, the dissolution amount of carbon dioxide is heavily influenced by the water depth of a culture vessel and/or the size of the diffused bubbles. Further, a mixed air containing 1-5% of carbon dioxide is often used as the gas to be diffused, and thus it is difficult to dissolve the carbon dioxide efficiently.

With a standard culture vessel having a shallow water depth of about 20-30 cm, it is generally difficult to retain carbon dioxide in culture solution until the carbon dioxide is sufficiently dissolved. Therefore, using one of the main dissolution methods, “a method of directly inserting an air diffuser into a culture vessel” may result in the dissolution efficiency of the input carbon dioxide of one-hundredth or less. Further, it is not desirable to release a large amount of undissolved carbon dioxide into the atmosphere due to the aspect of environmental protection. However, the amount of undissolved carbon dioxide released into the atmosphere will increase if the dissolution efficiency of carbon dioxide is low. Additionally, if the dissolution efficiency of carbon dioxide is low, the cultivation period will be prolonged due to the lack of carbon dioxide and thereby the costs of the cultivation will also increase.

To overcome the above deficiencies, a part of or the entire water depth of a culture vessel may be deepened to prolong the time period to retain carbon dioxide in the culture solution. In this case, however, the closer to the bottom surface of the culture vessel, the lower the culture efficiency due to insufficient light. Additionally, it may become difficult to install the culture vessel as the water depth of the culture vessel increases.

Alternatively, nanobubbles or microbubbles may be used as a high-efficiency dissolution method of carbon dioxide. However, aerating the micro-bubbled carbon dioxide into the culture solution will reduce the apparent absorbance of the culture solution. Therefore, it may not be suitable for algae cultivation.

It is also necessary to minimize a physical load on algae in order to avoid damaging the algae cells. To that end, it is desirable not to circulate the culture solution with a high pressure and high flowrate pump, not to generate swirling flow using such a pump, and not to generate bubbles with a Venturi tube. That is, although the importance of dissolving carbon dioxide in the culture solution has been recognized for algae cultivation, there is still a room to improve the dissolution method.

Hereinafter, a condition for inputting carbon dioxide and the dissolution efficiency of carbon dioxide with the gas dissolution device 3 of the first embodiment will be described.

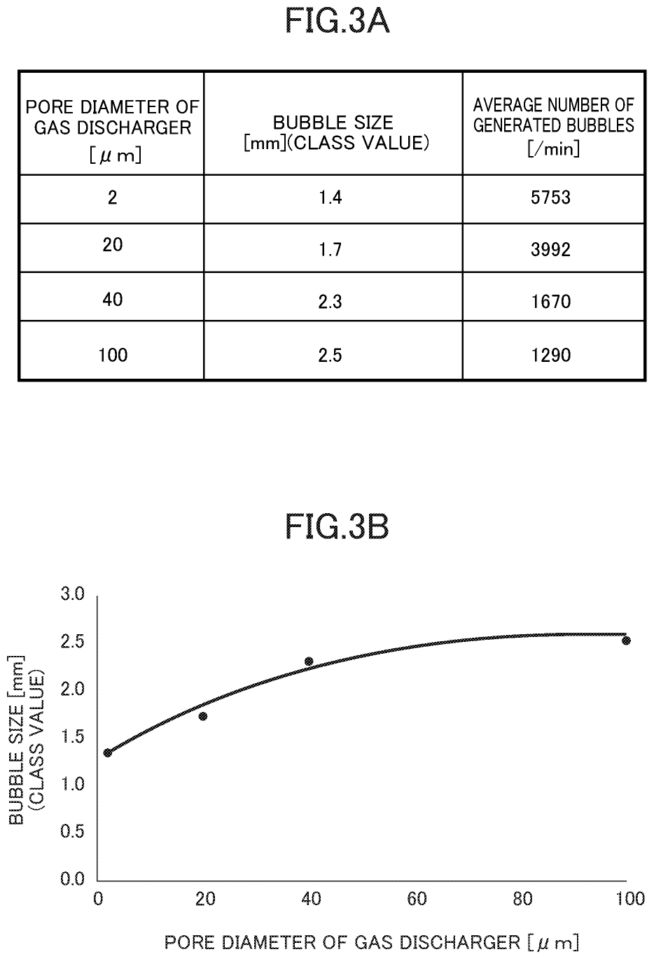

FIG. 3A and FIG. 3B show, in the gas dissolution device 3 of the first embodiment, relations of the pore diameters of the gas discharger 53, class values (hereinafter referred to as “bubble sizes”) of sphere-equivalent diameters of bubbles generated in relation to the pore diameters, and average numbers of the generated bubbles. As indicated by the relations shown in FIG. 3A and FIG. 3B, the larger the pore diameter of the gas discharger 53, the larger the bubble size generated. However, when the pore diameter exceeds 40 the bubble size remains about 2.5 mm regardless of the change in the pore diameter. FIG. 3C shows relations between the pore diameters of the gas discharger 53 and the average numbers of the generated bubbles. As shown in FIG. 3C, the average number of the generated bubbles also becomes almost constant when the pore diameter exceeds 40 μm. Accordingly, the pore diameters of the gas discharger 53 in the first embodiment are designed to 1-100 μm. With this, it is possible to obtain the bubbles having an appropriate size and an appropriate number without unnecessary increasing the pore diameters.

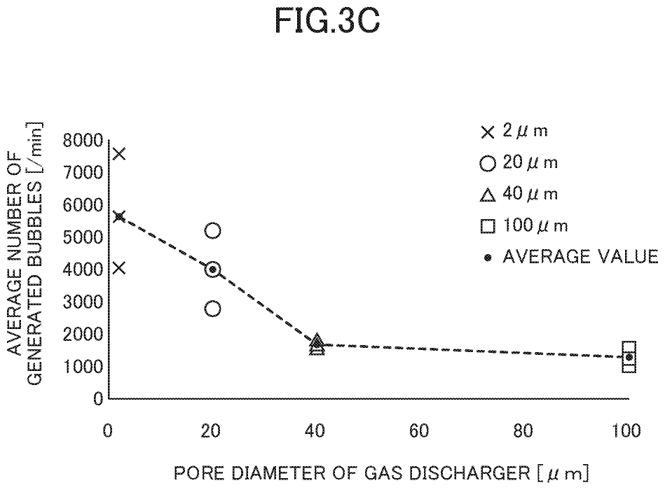

FIG. 4A shows, in the gas dissolution device 3 of the first embodiment, relations between the bubble sizes of the gas discharger 53 and the dissolution efficiencies of carbon dioxide. FIG. 4B shows, in the gas dissolution device 3 of the first embodiment, relations between the water depths H2 from the gas discharger 53 to the liquid levels 10a of the culture solution 100 stored in the dissolution vessel 10 and the dissolution efficiencies of carbon dioxide. The dissolution efficiencies are calculated in accordance with the following equation (1). It is known that a higher dissolution efficiency is preferable.

$$Dissolution Efficiency ( \% ) = \frac{Dissolved Amount}{Charged Amount} \times 100 \qquad ( 1 )$$

As indicated by the relations shown in FIG. 4A and FIG. 4B, the smaller the bubble size, the higher the dissolution efficiency, and thus is preferable. Additionally, it should be understood that as long as the water depth H2 is 450 mm or deeper, it shows preferable performance (i.e., having dissolution efficiency of 50% or higher) as the gas dissolution device 3. Accordingly, in the first embodiment, the water depth H2 to the liquid level 10a of the culture solution 100 stored in the dissolution vessel 10 is set to 450 mm or deeper. With this, it is possible to secure a required dissolution efficiency of carbon dioxide for the gas dissolution device 3.

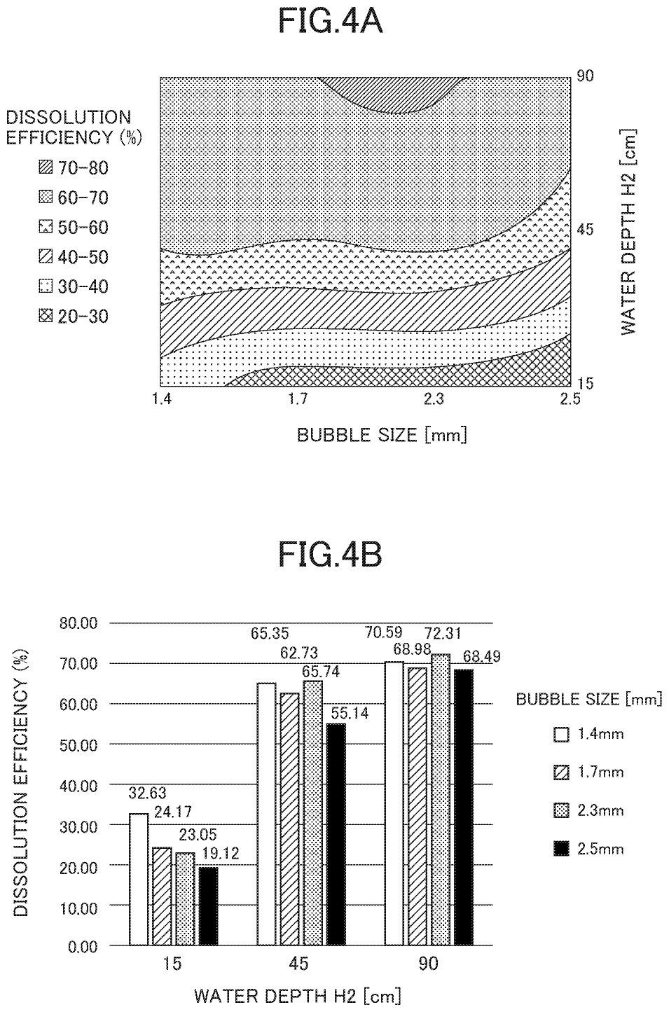

As shown in FIG. 5, when the bubble size is 1.4 mm, the dissolution efficiency reaches 50% or more by setting the water depth H2 somewhere between 300 mm and 350 mm. This also suggests that when the bubble size is 1.4 mm, the water depth H2 can be 350 mm or even deeper. Accordingly, in the first embodiment, the water depth H2 is set to 350 mm or deeper when the bubble size is 1.4 mm or smaller. With this, it is possible to secure a required dissolution efficiency of carbon dioxide for the gas dissolution device 3 even if the water depth H2 is shallow.

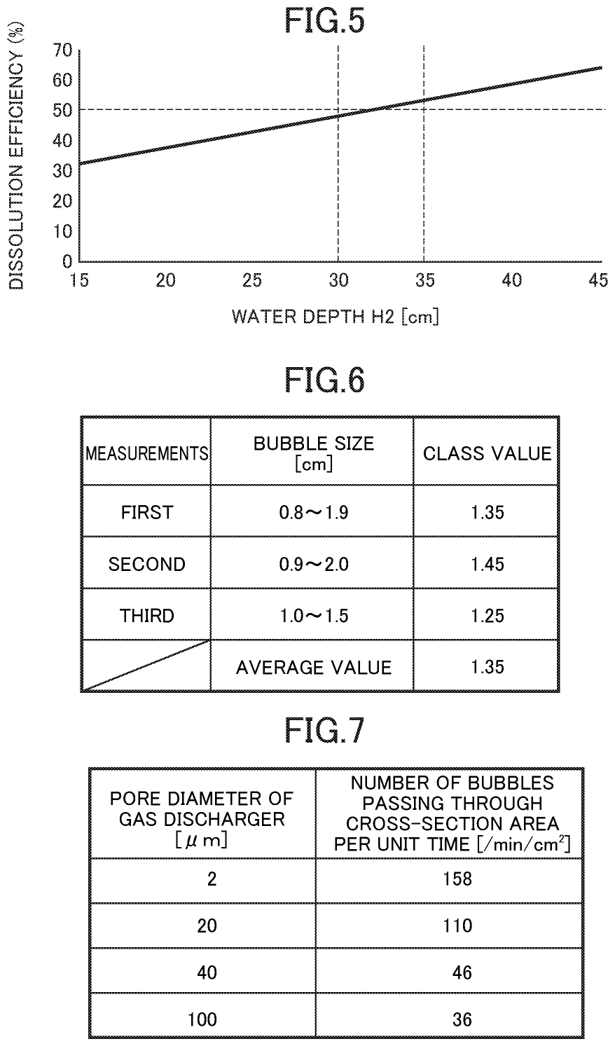

As shown in FIG. 3A to FIG. 4B, the pore diameter of the gas discharger 53 with the highest dissolution efficiency is 2 μm, and as shown in FIG. 6, the confirmable smallest bubble size generated by the gas discharger 53 having the pore diameter of 2 μm or smaller is 1.0 mm or smaller. Accordingly, the gas dissolution device 3 of the first embodiment is designed such that the gas discharger 53 generates carbon dioxide bubbles having the sphere-equivalent diameter (i.e., bubble size) of 2.5 mm or smaller, preferably 1.0 mm or smaller. With this, it is possible to secure a required dissolution efficiency when the water depth H2 is 450 mm or deeper, and it is possible to further improve the dissolution efficiency when the generated bubble size is 1.0 mm or smaller.

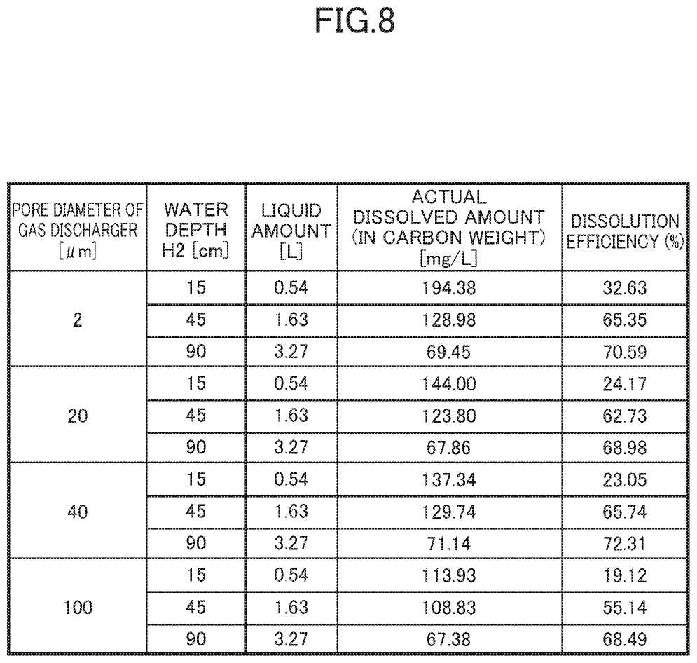

It is also possible to calculate the number of bubbles passing through a cross-section area per unit time based on the relation between the average number of the generated bubbles and the cross-section area of the dissolution vessel 10. The number of bubbles passing through the cross-section area per unit time varies depending on the size of the pore diameter of the gas discharger 53. That is, as shown in FIG. 7, the larger the pore diameter, the less the bubbles passing through the cross-section area. However, the number is 35/min/cm² or more. Accordingly, the gas discharger 53 of the first embodiment is designed such that the number of bubbles passing through the cross-section area per unit time is 35/min/cm² or more. With this, it is possible to secure the average number of the generated bubbles that can secure a required dissolution efficiency of carbon dioxide, thereby securing a required dissolution efficiency.

FIG. 8 shows relations of the pore diameters of the gas discharger 53, the water depths H2, the liquid amounts in the dissolution vessel 10, actual dissolved amounts of carbon dioxide (in terms of carbon weight), and dissolution efficiencies of carbon dioxide when the same amount of carbon dioxide is input for the same time period. As indicated by the relations shown in FIG. 8, the dissolution efficiencies are reduced when the actual dissolved amount is large. Theoretically, the more the dissolved amount of carbon dioxide in the culture solution 100, the release speed of carbon dioxide from the culture solution 100 increases. Hence, it is desirable to input carbon dioxide in order to maintain the dissolved amount of carbon dioxide in the culture solution 100 at a predetermined amount. Since the required dissolution efficiency of carbon dioxide is 50% or greater, it is understood, from the results shown in FIG. 8, the dissolved amount of carbon dioxide (in terms of carbon weight) should be 200 mg/L or less. Accordingly, in the first embodiment, the setting values of the pore diameter of gas discharger 53, of the water depth H2, and of the liquid amount stored in the dissolution vessel 10 are adjusted to achieve the dissolved amount of carbon dioxide (in terms of dissolved inorganic carbon weight) in the dissolution vessel 10 to be 200 mg/L or less. With this, it is possible to prevent unnecessary input of carbon dioxide and to dissolve the input carbon dioxide efficiently.

Hereinafter, results of carbon dioxide dissolution experiments with the gas dissolution device 3 of the first embodiment, a first comparative example, and a second comparative example will be described.

In the experiment, a raceway-type vessel shown in FIG. 1 is used as the culture vessel 2, and the liquid amount is set to 150 liters, the water depth H1 is set to 130-135 mm, and the stirring speed of liquid is set to 11 cm/sec. Further, an air diffusing tube is directly inserted into the liquid (tap water) stored in the culture vessel 2. As the gas dissolution condition of the first comparative example, 100% carbon dioxide was diffused at 60 mL/min (at 1 atm). As the gas dissolution condition of the second embodiment, an air mixed with 1% carbon dioxide was diffused at 6,000 mL/min (at 1 atm).

In the gas dissolution device 3 of the first embodiment, a constant amount (1-2 L/min.) of the liquid (tap water) is supplied to the dissolution vessel 10 (liquid amount of 5 liters) from the raceway-type culture vessel 2 (liquid amount of 150 liters) shown in FIG. 1. Additionally, 100% carbon dioxide is input at 60 mL/min. (at 1 atm) into the tap water supplied to the dissolution vessel 10 via the gas discharger 53. The tap water in which the carbon dioxide is dissolved in the dissolution vessel 10 is then returned to the culture vessel 2 through the monitoring vessel 71 (liquid amount of 1 liter) at a constant amount (1-2 L/min.).

In the experiment, the dissolved mount of carbon dioxide is measured in the culture vessel 2 as the dissolved inorganic carbon weight. In the gas dissolution device 3 of the first embodiment, when the pH value detected in the monitoring vessel 71 became a predetermined value or less, the supply of carbon dioxide was terminated.

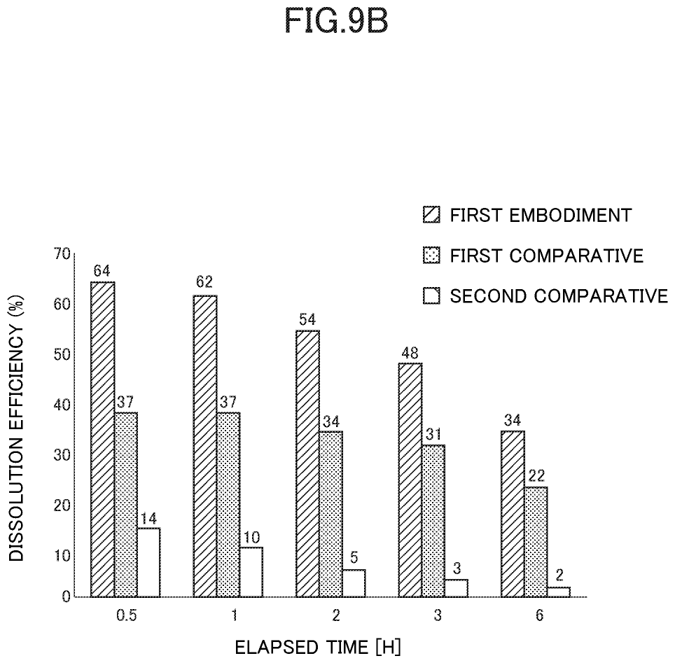

FIG. 9A and FIG. 9B show a summary of the experimental results and the deterioration in dissolution efficiency over time. As shown in FIG. 9A, the maximum dissolution efficiency with the gas dissolution device of the first comparative example was 37%, and the maximum dissolution efficiency with the gas dissolution device of the second comparative example was 14%. In contrary, the maximum dissolution efficiency with the gas dissolution device 3 of the first embodiment was 64%. With the gas dissolution device 3 of the first embodiment, the liquid stored in the culture vessel 2 is supplied to the dissolution vessel 10 and is returned to the culture vessel 2 after dissolving carbon dioxide in the dissolution vessel 10. During the process, the water depth H2 from the gas discharger 53 to the liquid level 10a of the liquid stored in the dissolution vessel 10 is set deeper than the water depth H1 of the liquid stored in the culture vessel 2. Due to the configuration, the dissolution efficiency of carbon dioxide is improved with a simple structure compared to the conventional carbon dioxide dissolution method in which an air diffuser is directly inserted into the culture vessel 2.

As shown in FIG. 9B, the dissolution efficiency of carbon dioxide decreases in any input conditions as the time passes since the start of the carbon dioxide dissolution experiment. However, it should be understood that the gas dissolution device 3 of the first embodiment can suppress the decrease in dissolution efficiency compared to the gas dissolution devices of the first comparative example and of the second comparative example. That is, the gas dissolution device 3 of the first embodiment can improve the dissolution efficiency of carbon dioxide and suppress the input amount of carbon dioxide. Further, the gas dissolution device 3 of the first embodiment can prevent carbon dioxide shortage in the culture solution 100 and shorten the cultivation period required for algae cultivation so as to reduce various costs for cultivation.

The gas dissolution device 3 of the first embodiment includes the first circulation pipe 20 that supplies the culture solution 100 stored in the culture vessel 2 to the dissolution vessel 10. The liquid ejection port 23 of the first circulation pipe 20 is positioned higher than the gas discharger 53 fixed to the other end of the gas supply pipe 50. Therefore, the culture solution 100 ejected from the first circulation pipe 20 flows down toward the bottom surface 11 of the culture vessel 2. On the other hand, the carbon dioxide discharged from the gas discharger 53 flows upward in the dissolution vessel 10.

Accordingly, the flow direction of the culture solution 100 supplied to the dissolution vessel 10 and the moving direction of carbon dioxide input to the dissolution vessel 10 are opposite to each other, thereby improving the dissolution efficiency of carbon dioxide.

The gas dissolution device 3 of the first embodiment includes the second circulation pipe 30 that returns the culture solution 100 stored in the dissolution vessel 10 to the culture vessel 2. The liquid suction port 33 of the second circulation pipe 30 is open to the side surface 12 of the dissolution vessel 10 and is positioned lower than the gas discharger 53. As described above, the carbon dioxide discharged through the gas discharger 53 flows upward in the dissolution vessel 10. Accordingly, the carbon dioxide discharged through the gas discharger 53 seldomly flows into the second circulation pipe 30. Therefore, it is possible to keep the input carbon dioxide in the dissolution vessel 10 in order to sufficiently dissolve the carbon dioxide, thereby further improving the dissolution efficiency of carbon dioxide.

Further, the gas dissolution device 3 of the first embodiment includes the pH monitor 70 that monitors the pH value of the culture solution 100 stored in the dissolution vessel 10. The mass flow controller 60 that controls the flowrate of the carbon dioxide input to the dissolution vessel 10 controls the carbon dioxide flowing through the gas supply pipe 50 based on the monitoring result of the pH monitor 70.

With this, it is possible to promptly detect the change in the pH value due to the change in the flowrate of the carbon dioxide. Accordingly, it is possible to appropriately control the input amount of carbon dioxide compared to the case where the flowrate of carbon dioxide input to the dissolution vessel 10 is controlled based on, for example, the pH value of culture solution 100 stored in the culture vessel 2.

Additionally, the pH monitor 70 includes the monitoring vessel 71 that communicates with the dissolution vessel 10 and the pH sensor 73 that measures the pH value of the culture solution 100 stored in the monitoring vessel 71. With this, the pH value of the culture solution 100 stored in the dissolution vessel 10 is measured outside the dissolution vessel 10, thereby preventing the bubbles of the carbon dioxide from adhering to the pH sensor 73. Accordingly, it is possible to suppress the occurrence of an error in the pH measurement and to improve the measurement accuracy of the pH value. Additionally, the culture solution 100 flown into the monitoring vessel 71 has an even carbon dioxide concentration since it is a liquid after dissolving the carbon dioxide. Therefore, it is possible to further suppress the occurrence of an error in the pH measuring and to improve the measurement accuracy of the pH value.

In the first embodiment, the height of the liquid level 71d of the culture solution 100 stored in the monitoring vessel 71 is equal to the height of the liquid level 10a of the culture solution 100 stored in the dissolution vessel 10. That is, the monitoring vessel 71 is provided at a position to allow the heights of the liquid levels 71d, 10a to be equal to each other. With this, it is possible to store the culture solution 100 up to the vicinity of each upper surface 13, 71d without overflowing the culture solution 100 from the corresponding vessel 10, 71. Therefore, it is possible to reduce the size of the device since there is no need to enlarge the dissolution vessel 10 and the monitoring vessel 71 unnecessarily.

In the gas dissolution device 3 of the first embodiment, the liquid discharge opening 36 is formed on the bottom surface 11 (bottom part) of the dissolution vessel 10, and the liquid discharge opening 36 is connected to the discharge pipe 35 having the switching valve 37. Once the switching valve 37 is open, the culture solution 100 in the dissolution vessel 10 flows into the discharge pipe 35 through the liquid discharge opening 36 and returns to the culture vessel 2 while bypassing the monitoring vessel 71. Accordingly, it is possible to discharge sediment such as algae that have been settled in the vicinity of the bottom surface 11 of the dissolution vessel 10 from the dissolution vessel 10 together with the culture solution 100.

The algae cultivation device 1 of the first embodiment uses the gas dissolution device 3 of the first embodiment to dissolve carbon dioxide in the culture solution 100. With this, it is possible to efficiently input carbon dioxide required for the algae cultivation to the culture vessel 2. Further, in the first embodiment, the carbon dioxide is dissolved in the culture solution 100 stored in the dissolution vessel 10, and then the culture solution 100 in which the carbon dioxide has been dissolved is returned to the culture vessel 2. Therefore, it is possible to prevent a sudden change in the pH value of the culture solution 100 stored in the culture vessel 2, and to prevent the bubbles of carbon dioxide from contacting the algae cells thereby suppressing damage on the algae cells in the culture solution 100.

Although the gas dissolution device and the algae cultivation device of the present disclosure have been described based on the first embodiment, it should not be limited thereto. It should be appreciated that variations or modifications may be made in the embodiment by persons skilled in the art without departing from the scope of the present invention as defined by the following claims.

In the first embodiment, the gas dissolution device 3 is exemplarily installed on a trolly with wheels. In this case, it is possible to move the gas dissolution device 3 as needed by disconnecting the one end 21 of the first circulation pipe 20 and the one end 31 of the second circulation pipe 30 from the culture solution 100 stored in the culture vessel 2. It is also possible to retrofit the gas dissolution device 3 to the culture vessel 2. However, it is not limited thereto. The first and the second circulation pipes 20, 30 may be fixed to the culture vessel 2 to integrate the gas dissolution device 3 and the culture vessel 2.

In the first embodiment, the culture vessel 2 and the dissolution vessel 10 are connected to each other through the first and the second circulation pipes 20, 30. Carbon dioxide is input to the culture solution 100 in the dissolution vessel 10, and the culture solution 100 is continuously circulated between the culture vessel 2 and the dissolution vessel 10 by the circulation mechanism 40. However, it is not limited thereto. The culture vessel 2 and the dissolution vessel 10 may be independently installed. In this case, a certain amount of culture solution 100 may be pumped out from the culture vessel 2 to the dissolution vessel 10, and the culture solution 100 may be pumped out from the dissolution vessel 10 to return to the culture vessel 2 after dissolving carbon dioxide to the culture solution 100 in the dissolution vessel 10.

In the first embodiment, the one end 35a of the discharge pipe 35 connected to the bottom surface 11 of the dissolution vessel 10 is connected to a part of the second circulation pipe 30 between the monitoring vessel 71 and the second pump 42. The culture solution 100 that has been settled at the bottom of the culture vessel 2 is flown into the discharge pipe 35 and is returned to the culture vessel 2 while bypassing the monitoring vessel 71. However, it is not limited thereto. For example, the one end 35a of the discharge pipe 35 may be inserted into a container such as a bucket, such that the sediment settled at the bottom of the culture vessel 2 is not returned to the culture vessel 2. Alternatively, the one end 35a of the discharge pipe 35 may directly be inserted to the culture vessel 2, such that the sediment is returned to the culture vessel 2 while bypassing the second pump 42. This configuration may be advantageous since it is possible to prevent the second pump 42 from being clogged by the sediment contained in the culture solution 100 flowing in the discharge pipe 35.

In the first embodiment, the water depth H2 from the gas discharger 53 to the liquid level 10a of the culture solution 100 stored in the dissolution vessel 10 is set to 450 mm or deeper. However, it is not limited thereto. Algae cultivation is often carried out in a relatively shallow environment where the water depth H1 of the culture vessel 2 is commonly set to about 200-300 mm, whereas the water depth H1 of the culture vessel 2 of the first embodiment is set to 130-135 mm. Therefore, the water depth H2 may be set to twice or more the water depth H1 of the culture solution 100 stored in the culture vessel 2.

In the first embodiment, the liquid stored in the culture vessel 2 or in the dissolution vessel 10 is used as the culture solution 100 in which microalgae are suspended, and carbon dioxide is used as the gas to be dissolved in the culture solution 100. However, it is not limited thereto. For example, oxygen, ozone, hydrogen, nitrogen, or the like may be dissolved in water. Alternatively, oxygen or another gas may be dissolved in industrial wastewater. Further, a plurality of gas dissolution devices 3 may be installed on the main vessel, namely the culture vessel 2.

### CROSS-REFERENCE TO RELATED APPLICATION

The present application is based on and claims priority to Japanese Patent Application No. 2018-201899, filed on Oct. 26, 2018, the disclosure of which is hereby incorporated by reference in its entirety.

1. A gas dissolution device, comprising:
a dissolution vessel that stores a part of liquid stored in a main vessel;
a gas supply pipe that is connected to a gas supply source and supplies gas through a tip part inserted into the liquid stored in the dissolution vessel;
a gas discharger that is provided at the tip part of the gas supply pipe and turns the gas supplied from the tip part into bubbles; and
a gas controller that controls a flowrate of the gas flowing in the gas supply pipe,
wherein a water depth from the gas discharger to a liquid level of the liquid stored in the dissolution vessel is set deeper than a water depth of the liquid stored in the main vessel.

2. The gas dissolution device according to claim 1, comprising:
a first circulation pipe that supplies the liquid stored in the main vessel to the dissolution vessel,
wherein a liquid ejection port of the first circulation pipe is provided at a position higher than the gas discharger.

3. The gas dissolution device according to claim 1, comprising:
a second circulation pipe that returns the liquid stored in the dissolution vessel to the main vessel,
wherein a liquid suction port of the second circulation pipe is provided at a position lower than the gas discharger.

4. The gas dissolution device according to claim 1, comprising:
a pH monitor that monitors a pH value of the liquid stored in the dissolution vessel,
wherein the gas controller controls the flowrate of the gas flowing in the gas supply pipe based on a monitoring result of the monitor.

5. The gas dissolution device according to claim 4,
wherein the pH monitor comprises a monitoring vessel that communicates with the dissolution vessel, and a pH sensor that measures a pH value of the liquid stored in the monitoring vessel.

6. The gas dissolution device according to claim 5,
wherein the monitoring vessel is installed such that a height of a liquid level of the liquid stored in the monitoring vessel is equal to a height of a liquid level of the liquid stored in the dissolution vessel.

7. The gas dissolution device according to claim 1,
wherein the dissolution vessel has a liquid discharge opening formed at a bottom part of the dissolution vessel, and
the liquid discharge opening is connected with a discharge pipe including a switching valve.

8. A algae cultivation device, comprising:
a culture vessel that stores culture solution for culturing algae; and
a gas dissolution device that dissolves carbon dioxide in the culture solution,
wherein the gas dissolution device comprises:
a dissolution vessel that stores a part of the culture solution stored in the culture vessel;
a first circulation pipe and a second circulation pipe that communicate between the culture vessel and the dissolution vessel;
a first pump that delivers the culture solution stored in the culture vessel to the dissolution vessel through the first circulation pipe;
a second pump that returns the culture solution stored in the dissolution vessel to the culture vessel through the second circulation pipe;
a gas supply pipe that is connected to a carbon dioxide source and supplies carbon dioxide through a tip part inserted into the culture solution stored in the dissolution vessel;
a gas discharger that is provided at the tip part of the gas supply pipe and turns the carbon dioxide supplied from the tip part into bubbles; and
a gas controller that controls a flowrate of the carbon dioxide flowing in the gas supply pipe,
wherein a water depth from the gas discharger to a liquid level of the culture solution stored in the dissolution vessel is set deeper than a water depth of the culture solution stored in the culture vessel.
